(19) United States
(12) Patent Application Publication
(10) Pub. No.: US 2019/0381263 A1
(43) Pub. Date: Dec. 19, 2019

(54) Inhalation Sensor Block, Exhalation Sensor Block and System

(71) Applicant: Carleton Technologies, Inc., Orchard Park, NY (US)

(72) Inventors: William D. Siska, Marilla, NY (US); Mark Harms, Orchard Park, NY (US); Jeffrey Brzuszkiewicz, Amherst, NY (US); Lucas Mesmer, Elma, NY (US)

(73) Assignee: Carleton Technologies, Inc., Orchard Park, NY (US)

(21) Appl. No.: 16/445,761

(22) Filed: Jun. 19, 2019

Related U.S. Application Data
(60) Provisional application No. 62/686,824, filed on Jun. 19, 2018.

(51) Int. Cl. A61M 16/00; A61M 16/06

(52) U.S. Cl. CPC A61M 16/0003; A61M 16/06; A61M 2016/0018; A61M 2230/435; A61M 2016/0039; A61M 2016/0042; A61M 2230/432; A61M 2016/0027

(57) ABSTRACT

A system containing a sensor suitable for pilot respiration inhalation gas data collection and a sensor suitable for pilot respiration exhalation gas data collection can be interfaced to a host computer or operate autonomously. The system or sensor components thereof can be used as a research tool for in-flight monitoring of physiologic effects on pilots' performance caused by the unique conditions faced during flight, in addition to human factors engendered during the course of their duty (fatigue, sleep loss, etc.).

### CROSS REFERENCE

This application claims the benefit of the filing date of U.S. Provisional Patent Application Ser. No. 62/686,824, filed Jun. 19, 2018, which is hereby incorporated by reference in its entirety.

### FIELD

The present disclosure relates to an inhalation sensor module, an exhalation sensor module, and system for near-real-time breath-by-breath analysis of gas flow.

### BACKGROUND

The art lacks a research tool for in-flight monitoring of the physiologic characteristics of pilots' performance caused by the unique conditions faced during flight, in addition to human factors engendered during the course of their duty (fatigue, sleep loss, etc.). Due to requirements of air worthiness, aircraft integration complexities, human factors, and the need for good repeatability, reliability, and accuracy across a spectrum of physiologic monitors, research programs are focused on a pilot-mounted set of sensors. To date, systems using gas sensors that were developed neither worked accurately or consistently, especially in high-humidity, air flow (exhalation-side). The current oxygen sensing technology using a Fast-Fourier Transform (FFT) to calculate the change in phase-shift of oxygen-quenched optical fluorescence of a Ruthenium Chloride (RuCl) detector or using the amplitude of the fluorescence is not appropriate for this application. Prior systems also contain a CO₂ sensor having similar performance shortfalls, thus failing to meet requirements for in-flight monitoring of pilot physiology.

### SUMMARY

In accordance with one aspect of the present disclosure, there is provided a sensor module for near-real-time breath-by-breath analysis of a gas stream, including:
a flow-through assembly including:
   a housing having a gas inlet and a gas outlet,
   a gas pressure sensor capable of sensing the pressure of the gas stream flowing through the housing,
   a gas temperature sensor capable of sensing the temperature of the gas stream flowing through the housing,
   a gas humidity sensor capable of sensing the humidity of the gas stream flowing through the housing,
   a cabin pressure sensor capable of sensing the air pressure outside the housing,
   a cabin temperature sensor capable of sensing the air temperature outside the housing,
   a 3-axis accelerometer capable of determining the motion of the sensor block,
   a real time clock, and
   a gas O₂ sensor including a robust fast reacting oxygen sensing media, wherein the O₂ sensor has a desired rapid response time capable of determining the ppO₂ concentration in the gas stream flowing through the housing; and
a computer, in data communication with each sensor, containing software capable of executing calibration curves and performing compensation calculations based upon the sensor data, to determine the ppO₂ in the gas stream flowing through the housing.

In accordance with another aspect of the present disclosure, there is provided a sensor module for near-real-time breath-by-breath analysis of a gas stream, including:
a flow-through assembly including:
   a housing having a gas inlet and a gas outlet,
   a gas pressure sensor capable of sensing the pressure of the gas stream flowing through the housing,
   a gas temperature sensor capable of sensing the temperature of the gas stream flowing through the housing,
   a gas humidity sensor capable of sensing the humidity of the gas stream flowing through the housing,
   a cabin pressure sensor capable of sensing the air pressure outside the housing,
   a cabin temperature sensor capable of sensing the air temperature outside the housing,
   a 3-axis accelerometer capable of determining the motion of the sensor block,
   a real time clock,
   a gas O₂ sensor including a robust fast reacting oxygen sensing media, wherein the O₂ sensor has a desired rapid response time capable of determining the ppO₂ concentration in the gas stream flowing through the housing, and
   a gas CO₂ sensor capable of determining the ppCO₂ concentration in the gas stream flowing through the housing; and
a computer, in data communication with each sensor, containing software capable of executing calibration curves and performing compensation calculations based upon the sensor data, to determine the ppO₂ in the gas stream flowing through the housing.

In accordance with another aspect of the present disclosure, there is provided a system for near-real-time breath-by-breath analysis of a gas stream, including:
an inhalation flow-through assembly including:
   a housing having a gas inlet and a gas outlet,
   a gas pressure sensor capable of sensing the pressure of the gas stream flowing through the housing,
   a gas temperature sensor capable of sensing the temperature of the gas stream flowing through the housing,
   a gas humidity sensor capable of sensing the humidity of the gas stream flowing through the housing,
   a cabin pressure sensor capable of sensing the air pressure outside the housing,
   a cabin temperature sensor capable of sensing the air temperature outside the housing,
   a 3-axis accelerometer capable of determining the motion of the sensor block,
   a real time clock, and
   a gas O₂ sensor including a robust fast reacting oxygen sensing media, wherein the O₂ sensor has a desired rapid response time capable of determining the ppO₂ concentration in the gas stream flowing through the housing;
an exhalation flow-through assembly including:
   a housing having a gas inlet and a gas outlet,
   a gas pressure sensor capable of sensing the pressure of the gas stream flowing through the housing,
   a gas temperature sensor capable of sensing the temperature of the gas stream flowing through the housing,
   a gas humidity sensor capable of sensing the humidity of the gas stream flowing through the housing,
   a cabin pressure sensor capable of sensing the air pressure outside the housing,
   a cabin temperature sensor capable of sensing the air temperature outside the housing,
   a 3-axis accelerometer capable of determining the motion of the sensor block,
   a real time clock,
   a gas O₂ sensor including a robust fast reacting oxygen sensing media, wherein the O₂ sensor has a desired rapid response time capable of determining the ppO₂ concentration in the gas stream flowing through the housing, and
   a gas CO₂ sensor capable of determining the ppCO₂ concentration in the gas stream flowing through the housing and
a computer, in data communication with each sensor, containing software capable of executing calibration curves and performing compensation calculations based upon the sensor data, to determine the ppO₂ in the gas stream flowing through the housings.

These and other aspects of the present disclosure will become apparent upon a review of the following detailed description and the claims appended thereto.

### BRIEF DESCRIPTION OF THE DRAWINGS

FIG. 1 is an embodiment of an exploded ISB Assembly;

FIG. 2 is an embodiment of an ISB Electrical Design and Assembly Block Diagram;

FIG. 3 is an embodiment of an ESB Mechanical Design and Assembly;

FIG. 4 is an embodiment of an ESB Electrical Design and Assembly Electrical Schematics;

FIG. 5 is an embodiment of a ppO₂ Sensor-Physical Implementation Summary;

FIG. 6 is an embodiment of a Flow Sensor Implementation of Gas Pressure Sensors and Flow Path;

FIG. 7 is an embodiment of a Gas Temperature Sensor and Humidity Sensor;

FIGS. 8A and 8B are an embodiment of an ESB Mechanical Design and Assembly;

FIG. 9 is an embodiment of a 3-axis Accelerometer;

FIGS. 10A and 10B are an embodiment of a ppCO₂ Sensor;

FIGS. 11A and 11B are an embodiment of components of a mask pressure sensor;

FIG. 12 is a graph showing two breaths at 0 ft. equivalent altitude;

FIG. 13 is a graph showing a series of breaths at 0 ft. equivalent altitude;

FIG. 14 illustrates a segment of breathing at 8,000 ft. altitude equivalent pre and post exercise;

FIG. 15 illustrates a segment of acceleration during exercise at 8000 ft. equivalent altitude;

FIG. 16 is a graph of oxygen concentrations and gas humidity of a subject breathing various altitude equivalents of oxygen;

FIG. 17 is a graph of luminescence intensity or decay time vs. oxygen content;

FIG. 18 is a graph of luminescence time constants vs. oxygen content;

FIG. 19 is a graph of curve fits;

FIG. 20 is a graph of “a” coefficients vs. temperature; and

FIG. 21 is a graph of “c” coefficients vs. temperature.

### DETAILED DESCRIPTION

The present disclosure relates to a system containing two sensor modules that can be interfaced to a host computer or operate autonomously. The system can be used as a research tool for in-flight monitoring of physiologic effects on pilots' performance caused by the unique conditions faced during flight, in addition to human factors engendered during the course of their duty (fatigue, sleep loss, etc.).

The Inhalation Sensor Block (ISB) is a sensor module suitable for pilot respiration inhalation gas data collection and is intended to be located directly in series with the pilot gas supply, post regulator. The ISB primarily operates as an autonomous standalone battery operated device storing sensor data on a micro SD Card (data storage). It can also be used to provide data query response to a host computer. In an embodiment, the hardware configuration of the ISB also includes a power switch, an on-board real time clock with back-up battery, a micro SD Card socket, and a “local” primary battery (9V). In an embodiment, the ISB preferably contains the following sensors: ppO₂; inhalation gas flow; inhalation gas temperature; inhalation gas humidity, inhalation (delivery) gas pressure; cabin pressure; cabin temperature; and 3-axis accelerometer. In an embodiment of the system design, the ISB is slave to the host computer. Other embodiments of the ISB with fewer components are suitable.

The Exhalation Sensor Block (ESB) is a sensor module suitable for pilot respiration exhalation gas data collection. This device may be mask mounted or connected to an exhalation tube. As with the ISB, the ESB primarily operates as an autonomous standalone battery operated device with a real-time clock and micro SD Card (data storage). The device(s) can also be used to provide data streaming to a host computer. In an embodiment, the ESB preferably contains the following sensors: ppO₂; exhalation gas flow; exhalation gas temperature; exhalation gas humidity; exhalation gas pressure; mask pressure; cabin pressure; cabin temperature; 3-axis accelerometer; and ppCO₂. In an embodiment of the system design, the ESB is slave to the host computer. Other embodiments of the ESB with fewer components are suitable.

The host computer serves as the communication master to collect data from the ISB, ESB or both and present this information to the diagnostician or pilot, etc. A suitable host computer may be responsible for the following functionality: synchronizing data collection (sensor readings) from the ISB and ESB; real time clock synchronization with the ISB and ESB; data display; and calibration utilities. The ISB and ESB can be used independently, or as a data collecting pair, or multiple sets of ISB and ESB devices can be used. In each scenario, data synchronization between units is maintained by time synchronization of each devices real-time clock to a common host computer's time clock and by each device independently time and date stamping each data collection sample.

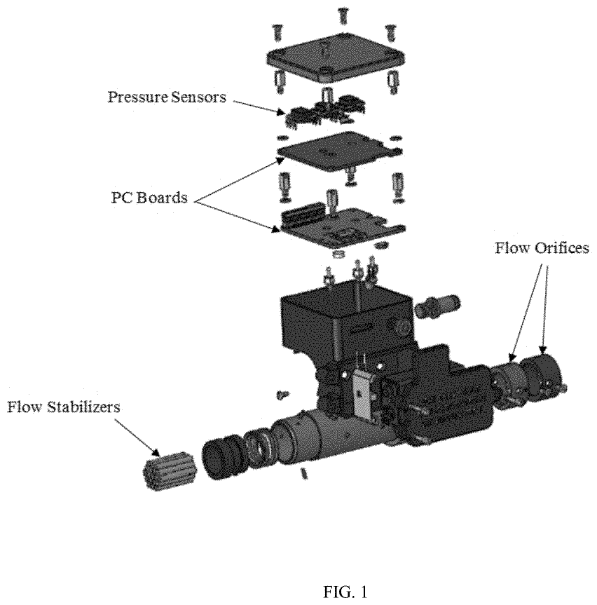

FIG. 1 is an exploded view of an ISB Assembly.

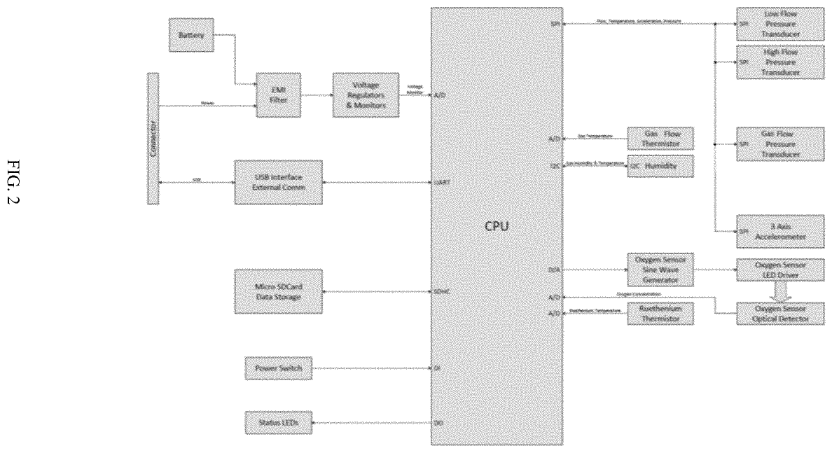

FIG. 2 is an ISB Electrical Design and Assembly Diagram. The capabilities of the ISB allow near-real-time breath-by-breath analysis of product gas air flow, gas (mask delivery) pressure, gas temperature, ppO₂, gas humidity, cabin pressure, cabin temperature, and 3-axis acceleration.

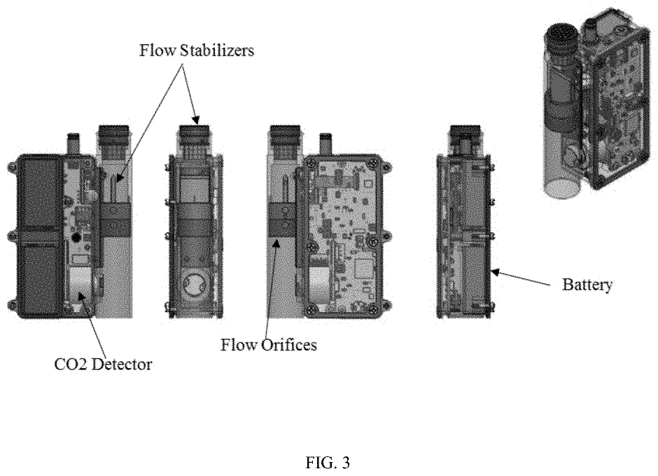

FIG. 3 is an ESB Mechanical Design and Assembly.

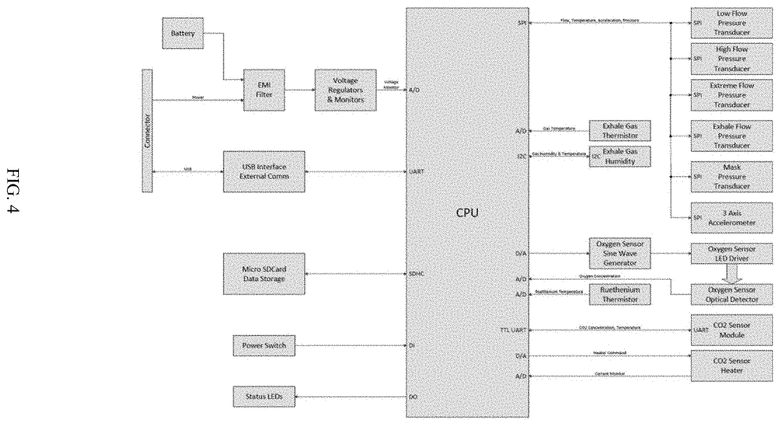

FIG. 4 is an ESB Electrical Design and Assembly Electrical Schematics. The capabilities of the ESB allow near-real-time breath-by-breath analysis of exhalation gas flow, exhalation gas pressure, exhalation gas temperature, ppO₂, exhalation gas humidity, cabin pressure, cabin temperature, 3-axis acceleration (for flow measurement compensation, ppCO₂, and mask pressure.

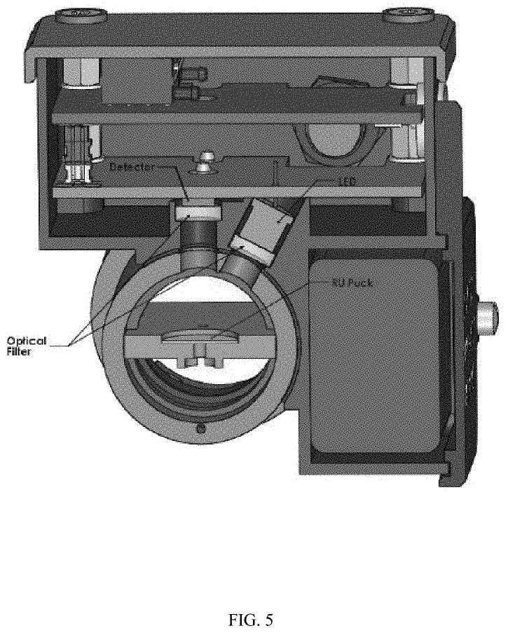

FIG. 5 is a ppO₂ Sensor which shows direct illumination of the sensing media “puck” target, minimized optical path free of (dichroic) mirrors, mechanically stable, geometry based on illumination dispersion angle of the LED, IR detector mounted directly at the center, and orthogonal, to the target puck, low sensing media thermal mass; temperature sensor in gas flow. Suitable O₂ sensors include a robust fast reacting oxygen sensing media. The O₂ sensor has a desired rapid response time. Prior to converting phase measurements to ppO₂ data, the phase measurement is preferably corrected for humidity.

A suitable oxygen sensor contains non-ruthenium based oxygen sensing media utilizing phase detection of the recoverable oxygen quenching fluorescence. This is characteristic of ruthenium, platinum or similar sensing compounds to measure partial pressure of oxygen. When stimulated with a particular wavelength of blue light these materials photo fluoresce at a particular wavelength of red-orange for a short time. The nominal persistence observed in an oxygen-free environment is a function of the composition of the sensing material. However, these compounds demonstrate recoverable quenching of the fluorescence amplitude and phase (decay rate) as a function of oxygen concentration. While both amplitude measurement and phase detection methods can be used, phase measurement involves fewer critical dependent factors, and is a preferred method for this application.

Sensitivity Factors

| | Amplitude | Phase |
|---|---|---|
| Factor | Measurement | Detection |
| Temperature | High | High |
| Pressure/Altitude | ppO2 | ppO2 |
| Humidity | High | High |
| Photo alignment (vibration) | High | Low |
| Contamination | High | Low |
| Aging (LED/photo-detector) | High | Moderate |
| Photo-bleaching | High | Low |

In an embodiment, the ppO₂ sensor, in its minimal implementation, is composed of a sinusoidal illumination source (LED), the sensing material, a photo sensor (photo diode), temperature sensor, and optical filtering. Implementation design considerations include:
a. Band-pass optical filtering is used at the “output” of the illumination source to restrict the wavelengths presented to the sensing material minimize to those required for photo florescence excitation and reject the thermal (IR) signature of the illumination source.
b. Each sensing material has an optimal phase differential response vs. stimulation frequency. The stimulation frequency is experimentally optimized.
c. Band-pass optical filtering is used at the “input” from the sensing material to restrict the wavelengths presented to the photo diode to those associated with the photo florescence response and minimize the effect of ambient light contamination.
d. The sensing material photo fluorescence phase response may be measured as the decay rate of an applied photo pulse or by measuring the sinusoidal phase change resulting change from continuous sinusoidal stimulation. Minimizing the harmonic content of the sinusoidal stimulus is critical. The electronic design uses multiple pole filtering to minimize the content of first harmonic by at least 64 dB.
e. Sensing material photo fluorescence response is dependent on the amplitude of photo stimulation. The electronic design utilizes precision current control of the illuminated element (LED) to minimize amplitude variation.
f. Sensing materials are subject to significant reduction in photo fluorescence response due to photo-bleaching associated aging. The electronic design minimizes the amount of incidence stimulation to achieve a minimum acceptable s/n ratio though precision nominal photo stimulus signal level and exposure duty cycle control. In addition, the sampling duty cycle is reduced during times of zero flow as determined by the gas flow sensor.
g. In a similar manner, variation in stimulation intensity occurs due to source (LED) aging and thermal effects are minimized by minimizing illumination amplitude and duty cycle control.
h. Phase measurement is determined differentially from the source rather than absolute phase change. This method provides compensation for circuit based fixed propagation and D/A sampling induced delays.
i. The sensing material fluorescence response is observed using a photo diode and a transimpedance amplifier. The response rate, bandwidth, sensitivity, and noise density is optimized by reverse biasing the photodiode and bootstrapping it with a JFET to reduce the effect of photodiode capacitance.
j. The fluorescence response of the sensing material is heavily dependent on temperature. Monitoring of the surface of the sensing material is needed. A direct contact temperature probe is used for materials exhibiting high thermal mass or by measuring air temperature at the sensor for those with low thermal mass.
k. The ppO₂ sensor application is capable of experiencing a wide range of oxygen concentrations over a similarly wide range of temperatures. Calibration of the sensor requires the sensor phase response be measured and characterized over all combinations of operating temperature and partial pressure of oxygen range. Calibration compensation for temperature is provided.
l. For sensing media not sensitive to humidity, the humidity sensors in the ISB and ESB are optional equipment as humidity data is not used to calibrate the oxygen sensor. For sensing media sensitive to humidity, some sensing materials exhibit a combined temperature-humidity effect, while for others the humidity effect is generally temperature independent. In such cases humidity data is used to calibrate the oxygen sensor. Typically, the gas stream in the ESB is saturated and 100% humidity is assumed.
m. The differential phase between the illumination source and the photo-diode signal is determined by simultaneous A/D sampling and applying a Goertzel FFT. Other implementations have used a traditional DFT or quadrature extraction methods. These are sensitive to fundamental frequency harmonics whereas the Goertzel is not. The standard Goertzel algorithm is as follows:

$$X [ k ] = {\sum }_{n = 0}^{N - 1} x ( n ) {e}^{- j 2 π k \frac{n}{N}}$$

Where N is the total number of samples taken of signal x[n] and k represents the integer (index) of the harmonic component in the DFT or number of samples per cycle.
In sampling domain, the process is defined as:

$$s [ n ] = x [ n ] + 2 cos ( \frac{2 π k}{N} ) s [ n - 1 ] - s [ n - 2 ]$$

And the DFT harmonic k content is defined as:

$${y}_{k} ( n ) = s ( n ) + {e}^{- j 2 π k \frac{n}{N}} s [ n - 1 ]$$

Some additional fidelity can be achieved through higher sampling rates. The sampling rate has a smaller effect on accuracy than the total number of cycles sampled. It is, however, critical that that full integer cycle sampling be performed. It was experimentally determined that a minimum ten samples per cycle provides adequate phase resolution.
n. Built In Test (BIT) health monitoring is provided for illumination signal, photo-diode output signal, temperature, phase calculation, signal noise, and sensor calibration (life).

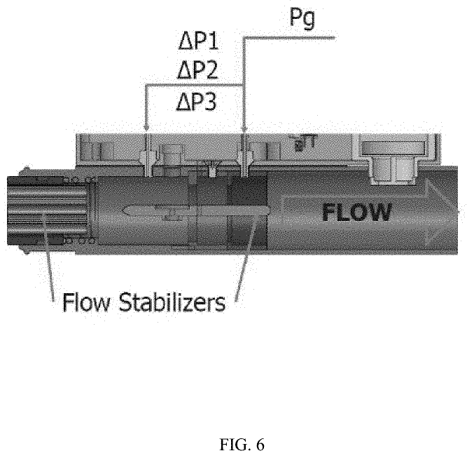

FIG. 6—shows an example of a suitable Flow Sensor: In order to achieve wide flow range (0-750 lpm) a series of orifices were used. Low flow (flow less than ˜175 lpm) ΔP₁+/−1 in-H₂O. High flow (flow between ˜175 lpm and ˜400 lpm) ΔP₂+/−5 in-H₂O. Extreme flow (ESB only) (flow greater than ˜400 lpm) ΔP₃+/−1 psi. Gas pressure Pg range: 0 to 1.6 bar. Gas temperature −30° C. to +63° C. Implementation considerations include:

a. Flow stabilizers and bifurcation is utilized to reduce turbulence at high flows.
b. The flow measurement is determined from the differential pressure across the orifice(s) and gas density. Gas density is calculated from the gas temperature and absolute pressure.
c. Gas temperature is measured using a thermistor suspended in the air flow path.
d. Differential and absolute pressure is measured using Integrated Circuit MEMs type miniature pressure sensors. These devices may be constructed using MEMs diaphragm and strain sensors that are orientation and acceleration sensitive. Compensation for orientation and acceleration is provided using the data from the accelerometer.
e. IAW with the ideal gas laws, the density of the sample gas is a function of the specific heat. The specific heat ratio of air is used. Alternatively, the specific heat of a nitrogen/oxygen mix as measured by the ppO₂ sensor is to be implemented in future versions.
f. BIT health monitoring is provided for microprocessor A/D failure, differential pressure range, absolute pressure range, temperature, pressure sensor device failure, and unstable flow conditions.

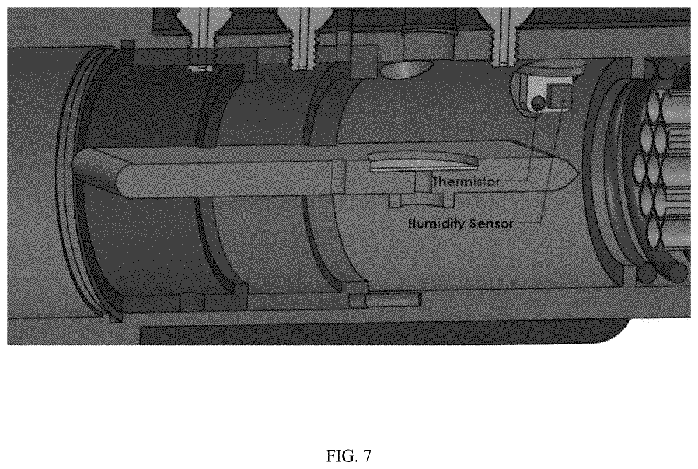

FIG. 7—Gas Temperature range −30° C. to 63° C., accuracy ±5% max FS, response less than 4 seconds, linearity less than 5%, sample at 200 Hz. Implementation Honeywell Series 112, 10 K±20% thermistor, resistance bridge with precision voltage reference, bridge optimization for calibration measurement range, located directly in air-flow path. Humidity Sensor Range from 0 to 100% RH, accuracy ±5% max. FS, response <10 sec, linearity <2%, sample rate 1 Hz. Implementation STMicroelectronics HTS221 capacitive sensor, 0 to 100% RH ±5% from 0° C. to 60° C., response ˜10 sec to 63% final value of R.H. step change, factory calibrated, located directly in air-flow path, internal heater for condensation recovery.

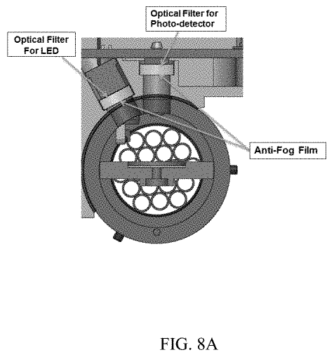

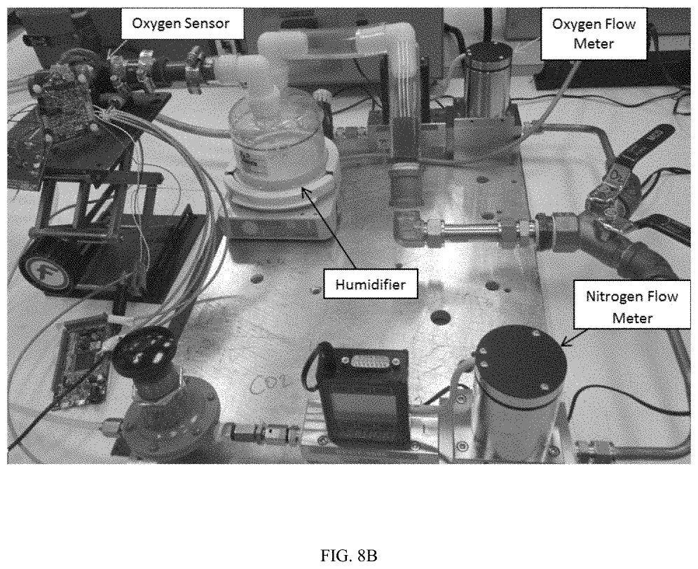

FIGS. 8A and 8B represent Oxygen Sensor Testing with 99% RH, 20% Oxygen, 10 LPM continuous flow with humidity control for ppO₂ sensor.

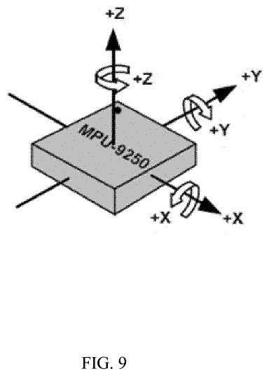

FIG. 9 is a 3-axis accelerometer range ±10 G, accuracy ±5% max. FS, response less than 5 msec, linearity less than 5%, sample rate 100 Hz. Implementation.

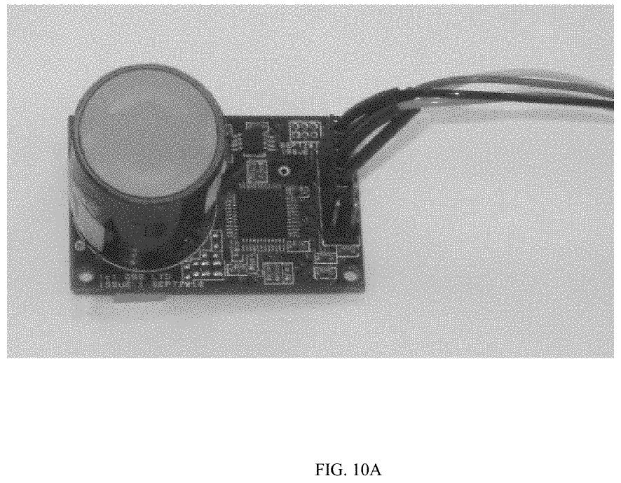

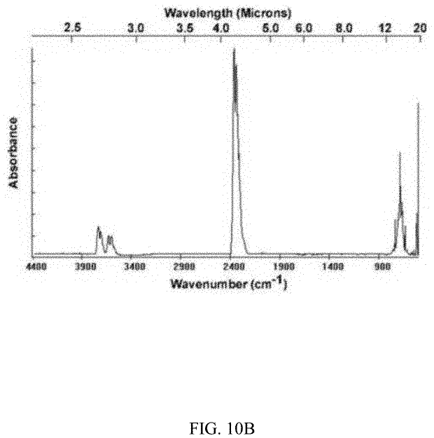

FIGS. 10A and 10B represent ppCO₂ sensor Range 0-152 mmHg, calibration range 20° C. to 35° C., accuracy ±5% typ. FS, response less than 100 msec, linearity less than 5%, sample at 20 Hz. Power input 3.2 to 5 Volts DC, power consumption 35 mW, the CO₂ sensor uses non-dispersive infrared absorbance (NDIR) to monitor CO₂, sensor is adversely affected by condensed moisture on internal reflecting surfaces.

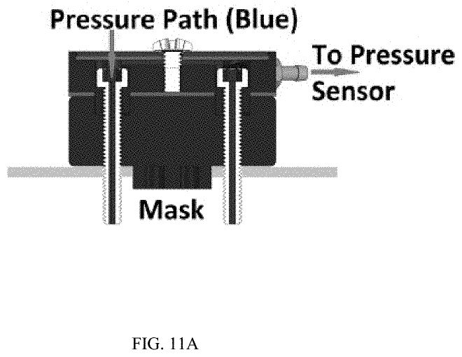

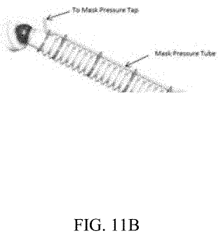

FIGS. 11A and 11B represent mask pressure sensor range ±240 mmHg, calibration range 2° C. to 51° C., accuracy ±2% typ. FS, response less than 40 msec, linearity less than 5%, sample at 200 Hz.

The disclosure will be further illustrated with reference to the following specific examples. It is understood that these examples are given by way of illustration and are not meant to limit the disclosure or the claims to follow.

### Example 1

Testing of the ISB and ESB was conducted at Cleveland State University (CSU). The university is equipped with a Reduced Oxygen Breathing Device (ROBD) that provides breathing gas to a subject at controlled oxygen concentrations to simulate breathing at altitude. Subjects were required to breathe from a mask while being exposed to various concentrations of oxygen under a series of external stressors. Subjects were monitored by medical professionals throughout testing.

The ISB contained a flow-through assembly including: a housing having a gas inlet and a gas outlet, a gas pressure sensor, gas temperature sensor, gas humidity sensor, cabin, cabin temperature sensor, 3-axis accelerometer, clock, and gas O₂ sensor including a robust fast reacting oxygen sensing media; and a computer, in data communication with each sensor, containing software for executing calibration curves and performing compensation calculations based upon the sensor data, wherein humidity data was used to calibrate the oxygen sensor.

The ESB contained a flow-through assembly including: a housing having a gas inlet and a gas outlet, a gas pressure sensor, gas temperature sensor, gas humidity sensor, cabin, cabin temperature sensor, 3-axis accelerometer, clock, gas CO₂ sensor, and gas O₂ sensor including a robust fast reacting oxygen sensing media; and a computer, in data communication with each sensor, containing software for executing calibration curves and performing compensation calculations based upon the sensor data, wherein humidity data was used to calibrate the oxygen sensor.

The ROBD was pneumatically plumbed thru a ¾″ tube to the ISB. Approximately 18″ of ¾″ tubing connected the ISB to the inlet of GENTEX's MBU-20/P oxygen mask. The subject interfaced directly with the mask and was fitted to ensure a tight seal was maintained. The ESB was attached to the exhalation valve of the MBU-20/P where expired gas from the user was vented to atmosphere. The following test protocols were observed:

Protocol A) Breath by Breath Breathing under the following conditions.

| Duration | Equivalent | |
|---|---|---|
| (s) | Altitude (ft) | Stressor |
| 300 | 0 | None |

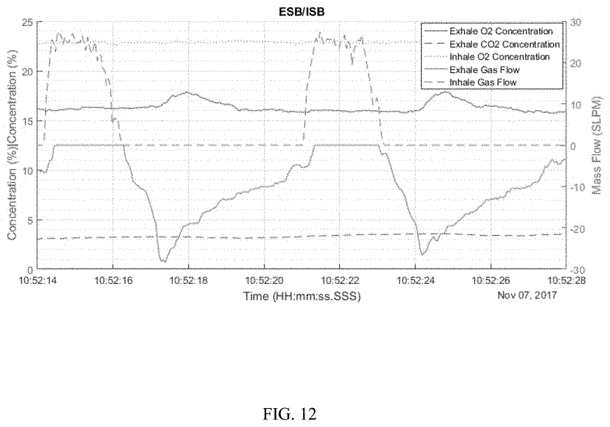

FIG. 12 illustrates a segment of the breath by breath test showing two breaths of breathing at 0 ft. equivalent altitude.

Measurements from ESB Sensors
Carbon dioxide
Oxygen
Flow

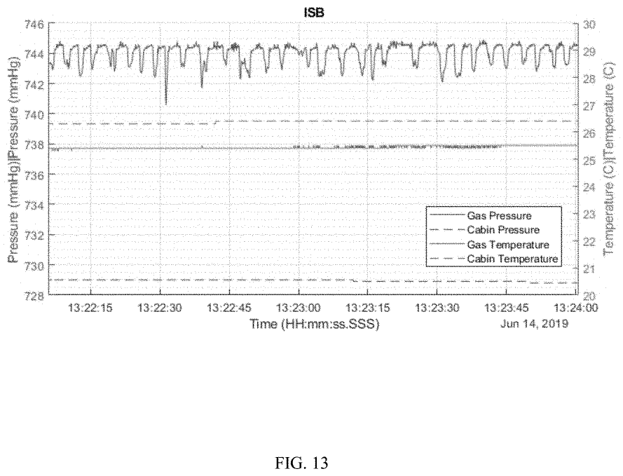

Measurements from ISB Sensors
Oxygen
Flow
   FIG. 13 illustrates a segment of the breath by breath test showing a series of breaths at 0 ft. equivalent altitude.

Measurements from ISB Sensors
Gas Pressure
Gas Temperature
Cabin Pressure
Cabin Temperature

Protocol B) Breathing at Altitude Under Stress
under the following conditions.

| Duration | Equivalent | |
|---|---|---|
| (s) | Altitude (ft) | Stressor |
| 60 | 8,000 | None |
| 30 | 8,000 | Exercise (repeated squats) |
| 150 | 8,000 | None |
| 30 | 8,000 | Exercise (repeated squats) |
| 150 | 8,000 | None |

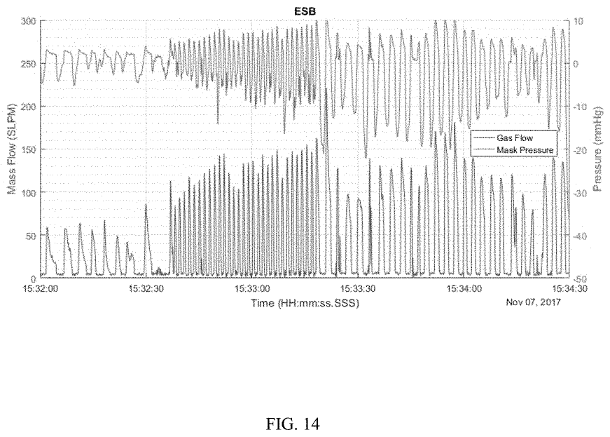

FIG. 14 illustrates a segment of breathing at 8,000 ft. altitude equivalent pre and post exercise.

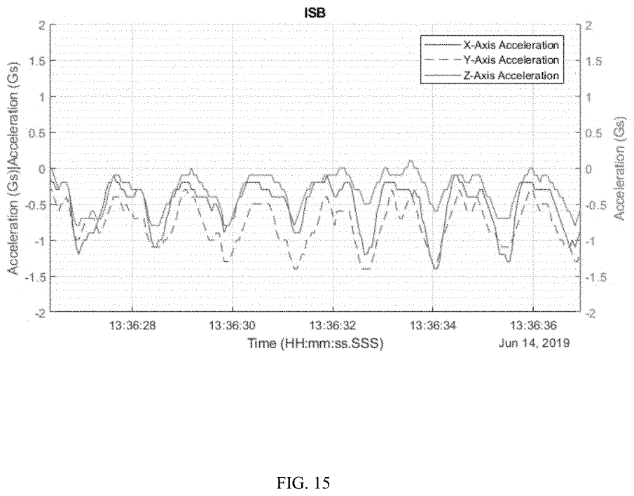

Measurements from ESB Sensors
Mask Pressure
Flow
   FIG. 15 illustrates a segment of acceleration during exercise at 8000 ft. equivalent altitude.

Measurements from ISB
Acceleration, X, Y, Z

Protocol C) Oxygen Concentrations at Varying Altitudes.

| Duration | Equivalent | |
|---|---|---|
| (s) | Altitude (ft) | Stressor |
| 120 | 600 | None |
| 90 | 8,000 | None |
| 30 | 600 | None |
| 90 | 18,000 | None |
| 30 | 600 | None |
| 90 | 25,000 | None |
| 30 | 600 | None |
| 15 | — | 100% Oxygen |

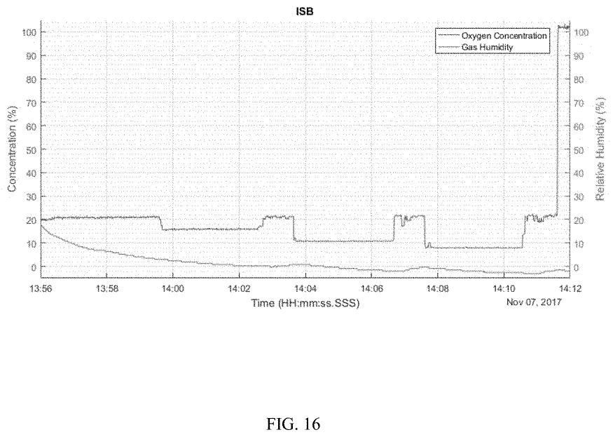

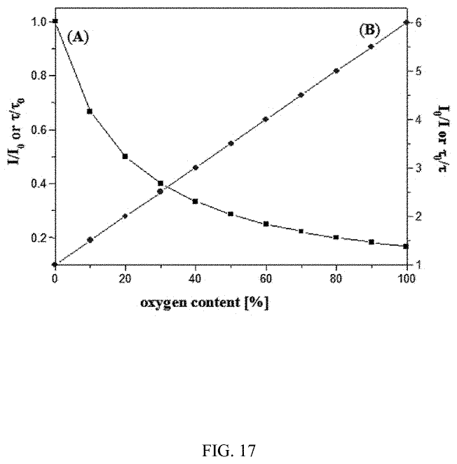

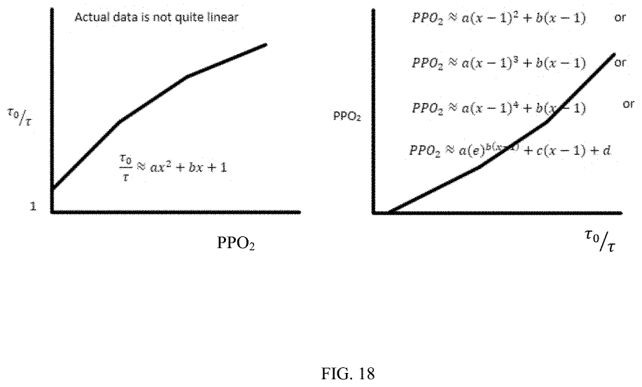

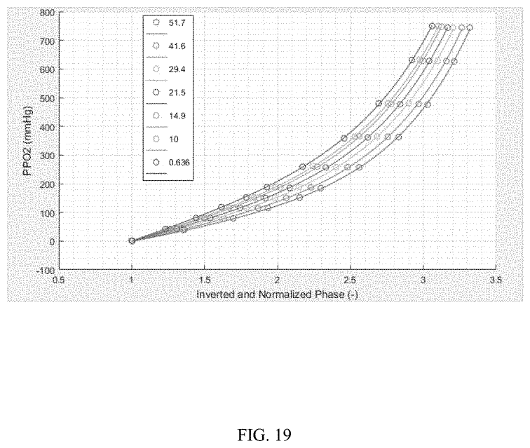

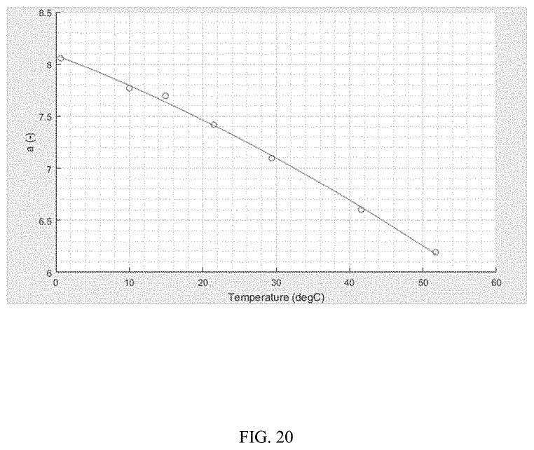

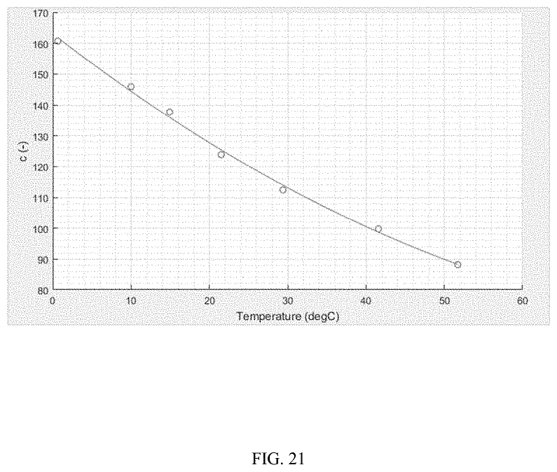

FIG. 16 is a graph of oxygen concentrations and gas humidity of a subject breathing various altitude equivalents of oxygen.

Measurements from ISB Sensors
Oxygen Concentration
Humidity

### Example 2

The present example relates to the calibration of an oxygen sensor commercially available from Ocean Optics, Largo Fla. containing non-ruthenium based oxygen sensing media for use in ISB and ESB software.

Note: The ppO₂ mmHg calculation is bounded by 0 mmHg to 760 mmHg.

The calibration is based on a non-ideal application of the Stern-Volmer Relationship. Stern-Volmer characterizes the relationship between ppO₂ and the fluorescing response of intramolecular deactivation (quenching) that occurs where the presence of one chemical can accelerate the decay rate of another chemical in its excited state.

$$\frac{{I}_{0}}{I} = \frac{{τ}_{0}}{τ} = 1 + {k}_{sv} [ {O}_{2} ]$$

### Where:

I=luminescence intensity in the presence of O₂
I₀=luminescence intensity in the absence of O₂
τ=luminescence decay time in the presence of O₂
τ₀=luminescence decay time in the absence of O₂
kₛᵥ=Stern-Volmer constant (quantifies the efficiency and therefore the sensitivity of the sensor)

Luminescence (intensity and decay time) decreases in the presence of oxygen

Ideal (theoretical) Stern-Volmer Plot

Experimentation confirms that as ppO₂ increases that intensity decreases, however due to the implementation of the Goertzel phase angle calculation it is observed that the phase angle increases and is 180° from the theoretical response.

The non-ideal is non-linear.

As noted, there are only two (identified) factors for determining phase-based calculation of Ocean Optics material phase-based calculation of ppO₂; temperature and excitation signal response delay.

Measure and record the phase response at various temperatures with ppO₂ concentrations varying from 0 mmHg to −760 mmHg. Invert Goertzel calculation phase angles:

phase=180°−phase

Calculate the average 0 mmHg ppO₂ phase value.

zero=mean[f(T,0 mmHg)]

Normalize the phase angles:

$$p = \frac{zero}{phase}$$

For each temperature, fit a curve of the ppO₂ vs. phase response (inverted and normalized phase angles) using the exponential equation of form:

ppO2=ae^{b(p-1)}+c(p−1)+d

Where: a=f(T)
b=f(T)
c=f(T)
d=f(T)
are all functions of temperature (° C.).

Calculate the average b and d coefficients from all of the temperature data sets.

Re-fit the ppO₂ vs. phase response (inverted and normalized phase angles) using the fixed b and d coefficients:

ppO2=ae^{[b(p-1)]}+c(p−1)+d

Where: a=f(T)
b=constant
c=f(T)
d=constant

Fit a curve of the “a” coefficients vs. temperature using a quadratic equation form:

a=a₁T²+a₂T+a₃

Fit a curve of the “c” coefficients vs. temperature using a quadratic equation form:

c=c₁T²+c₂T+c₃

The temperature compensated ppO₂ level can, therefore be calculated as:

a=a₁T²+a₂T+a₃

Where: a1, a2, a3=“a” term calibration constant (OPA1)
T=temperature (° C.)

c=c₁T²+c₂T+c₃

Where: c1, c2, c3=“c” term calibration constant (OPC1)

phase=180°−(phase+tare)

Where: tare=phase tare constant (OTAR see 2.5.1.8)

$$p = \frac{zero}{phase}$$

Where: zero=“zero” term calibration constant (OPZ0)

ppO2=ae^{[b(p-1)]}+c(p−1)+d

Where: b=“b” term calibration constant (OPB1)
d=“d” term calibration constant (OPD1)
Default values are:

### OPA1=−0.00018522-0.027414 8.0866

### OPB1=1.9285

### OPC1=0.010112-1.9677 163.06

### OPD1=−7.5407

### OPZ0=52.769

The data collection and phase sampling method for the Ocean Optics material is as follows:

Begin excitation.

The DAC generated 5 KHz square wave is filtered by an eighth order 7 KHz Butterworth filter resulting in a sine wave with very low harmonic content. The amplitude and offset will define the LED drive current level. The sine wave is non-zero, meaning that the LED output must never reach zero so that there is always a sensor excitation response. A multi-pole analog filter will convert the square wave into an acceptable sinusoid. It is important that the peak DAC output be bounded such that the various gains used in the individual filter stages do not create a signal clipping issue. This is also critical the photo-diode transimpedance amplifier output never reaches the (either) rail (or the output clips). While amplitude and phase of the response will vary with temperature and ppO₂, the amplitude response is not critical other than to provide sufficient signal discrimination. However, to simplify temperature correction, temperature is assumed to be constant over the sampling period.

No phase jitter in the DAC excitation frequency is permitted.

Begin ADC (sensor response) data sampling.

Synchronize ADC data sampling to DAC signal generator. Synchronization can be achieved through careful implementation of the ADC and DAC functions or by simultaneous sampling of the DAC output and sensor (ADC) response. If the simultaneous sampling method is used, both “channels” of data must be processed and software filtered identically. For discussion purposes, this example assumes hardware synchronization will be used. In either case, the sampling rate must be an even integer multiple of the DAC frequency. Fixed phase (time delay) between the DAC and sampling clock is permitted. However, sampling clock-to-DAC phase jitter is not permitted.

Store data in a single array.

Disable the LED excitation current (DAC output=0). Turn off the LED.

Measure the air flow (sensor) temperature. The time constant for the thermistor is very slow with respect to the sampling period and can be assumed to be constant for the sampling period.

Apply the Goertzel FFT algorithm to determine phase and amplitude. See the MATLAB code sample below.

Validate the signal. Sufficient amplitude and appropriate distance from the op-amp rails is required. Save the data even if the signal is unacceptable. If the signal amplitude is too small issue the SIGL BIT fault. If the signal amplitude is too large and risks peak clipping, set the SIGH BIT. However, if the data is okay, save the amplitude, raw phase and temperature in a 20 event deep data FIFO (1 second minimum of data) for diagnostic purposes.

Verify the phase measurement against acceptable phase range (not all phase angles are expected or permitted) and the phase stability (large instantaneous swings in phase are not expected nor permitted).

Calculate ppO₂ and limit the value between zero and the current absolute inhalation (delivery) gas pressure value. Filter the temperature and ppO₂ measurement through the median filter and exponential low pass digital filter. This value will be used as the reported ppO₂ output.

Tally the number of seconds the sensor target has been illuminated. Also integrate the illumination time multiplied by the ppO₂. These two values are key indicators for useful life before recalibration.

If the ppO₂ calculation results in “bad” data, even though the sensor may have been illuminated, no time is to be accumulated. The alternative is to “guess” at the ppO₂ value and assume a life impact. The premise for the “ZERO” is that calculation errors should never occur very often and be eliminated during development testing.

Repeat steps 1-10 at a rate of 1 to 20 times per second (target: LED ˜36% duty cycle at 5 KHz), nominal 20 Hz.

Although various embodiments have been depicted and described in detail herein, it will be apparent to those skilled in the relevant art that various modifications, additions, substitution, and the like can be made without departing from the spirit of the disclosure and these are therefore considered to be within the scope of the disclosure as defined in the claims which follow.

What is claimed:

1. A sensor module for near-real-time breath-by-breath analysis of a gas stream, comprising:
a flow-through assembly comprising:
a housing having a gas inlet and a gas outlet,
a gas pressure sensor capable of sensing the pressure of the gas stream flowing through the housing,
a gas temperature sensor capable of sensing the temperature of the gas stream flowing through the housing,
a cabin pressure sensor capable of sensing the air pressure outside the housing,
a cabin temperature sensor capable of sensing the air temperature outside the housing,
a 3-axis accelerometer capable of determining the motion of the sensor block,
a real time clock, and
a gas O₂ sensor comprising a robust fast reacting oxygen sensing media, wherein the O₂ sensor has a desired rapid response time capable of determining the ppO₂ concentration in the gas stream flowing through the housing; and
a computer, in data communication with each sensor, containing software capable of executing calibration curves and performing compensation calculations based upon the sensor data, to determine the ppO₂ in the gas stream flowing through the housing.

2. The sensor module according to claim 1, wherein the oxygen sensing media comprises a non-ruthenium based sensing media capable of use in a rapidly responding oxygen concentration measurement device.

3. The sensor module according to claim 1, wherein the gas O₂ sensor utilizes phase detection to determine the ppO₂ concentration.

4. The sensor module according to claim 1, wherein the gas pressure sensor comprises a plurality of mechanical orifices and uses the pressure differential across the orifices to measure the gas pressure.

5. The sensor module according to claim 1, further comprising a gas CO₂ sensor capable of determining the ppCO₂ concentration in the gas stream flowing through the housing.

6. The sensor module according to claim 1, further comprising a gas humidity sensor capable of sensing the humidity of the gas stream flowing through the housing.

7. The sensor module according to claim 6, wherein humidity data is used to calibrate the oxygen sensor.

8. A system for near-real-time breath-by-breath analysis of a gas stream, comprising:
an inhalation flow-through assembly comprising:
a housing having a gas inlet and a gas outlet,
a gas pressure sensor capable of sensing the pressure of the gas stream flowing through the housing,
a gas temperature sensor capable of sensing the temperature of the gas stream flowing through the housing,
a cabin pressure sensor capable of sensing the air pressure outside the housing,
a cabin temperature sensor capable of sensing the air temperature outside the housing,
a 3-axis accelerometer capable of determining the motion of the sensor block,
a real time clock, and
a gas O₂ sensor comprising a robust fast reacting oxygen sensing media, wherein the O₂ sensor has a desired rapid response time capable of determining the ppO₂ concentration in the gas stream flowing through the housing;
an exhalation flow-through assembly comprising:
a housing having a gas inlet and a gas outlet,
a gas pressure sensor capable of sensing the pressure of the gas stream flowing through the housing,
a gas temperature sensor capable of sensing the temperature of the gas stream flowing through the housing,
a cabin pressure sensor capable of sensing the air pressure outside the housing,
a cabin temperature sensor capable of sensing the air temperature outside the housing,
a 3-axis accelerometer capable of determining the motion of the sensor block,
a real time clock,
a gas CO₂ sensor capable of determining the ppCO₂ concentration in the gas stream flowing through the housing, and
a gas O₂ sensor comprising a robust fast reacting oxygen sensing media, wherein the O₂ sensor has a desired rapid response time capable of determining the ppO₂ concentration in the gas stream flowing through the housing; and
a computer, in data communication with each sensor, containing software capable of executing calibration curves and performing compensation calculations based upon the sensor data, to determine the ppO₂ in the gas stream flowing through the housings.

9. The system according to claim 8, wherein the oxygen sensing media comprises a non-ruthenium based sensing media capable of use in a rapidly responding oxygen concentration measurement device.

10. The system according to claim 8, wherein the gas O₂ sensor utilizes phase detection to determine the ppO₂ concentration.

11. The system according to claim 8, wherein the gas pressure sensor comprises a plurality of mechanical orifices and uses the pressure differential across the orifices to measure the gas pressure.

12. The system according to claim 8, wherein at least one of the inhalation flow-through assembly housing further comprises a gas humidity sensor capable of sensing the humidity of the gas stream flowing through the inhalation flow-through assembly housing and the exhalation flow-through assembly housing further comprises a gas humidity sensor capable of sensing the humidity of the gas stream flowing through the exhalation flow-through assembly housing.

13. The system according to claim 12, wherein humidity data is used to calibrate the oxygen sensor.
